(19) United States
(12) Patent Application Publication
(10) Pub. No.: US 2025/0352390 A1
(43) Pub. Date: Nov. 20, 2025

(54) CANNULA CLEARANCE

(71) Applicant: Alcon Inc., Fribourg (CH)

(72) Inventor: Michael Sam Cardamone, Keller, TX (US)

(21) Appl. No.: 19/184,144

(22) Filed: Apr. 21, 2025

Related U.S. Application Data
(60) Provisional application No. 63/647,723, filed on May 15, 2024.

(51) Int. Cl. A61F 9/007

(52) U.S. Cl. CPC A61F 9/00736

(57) ABSTRACT

An apparatus for eye surgery, the apparatus comprising a probe having a tube, a cutting edge disposed within the tube, and a stop, and a cannula having a first end configured to receive the tube and a second end adapted for insertion into an eye. The stop can be configured to position the cutting edge adjacent to the second end of the cannula when the tube is inserted into the cannula.

### TECHNICAL FIELD

The invention set forth in the appended claims relates generally to ophthalmic surgery, including, without limitation, systems, apparatuses, and methods for reducing or clearing cannula obstructions.

### BACKGROUND

The human eye can suffer a variety of maladies causing mild deterioration to complete loss of vision. While contact lenses and eyeglasses can compensate for some ailments, ophthalmic surgery may be required for others. For example, retinal detachment, traction retinal detachment, and trauma remain major causes of visual loss worldwide, despite continuing advances in vitreoretinal care, and pars plana vitrectomy is a leading management modality for the treatment for such conditions.

While the benefits of vitrectomy and other ophthalmic surgical procedures are known, improvements to surgical systems, components, and processes can continue to improve outcomes and benefit patients.

### BRIEF SUMMARY

New and useful systems, apparatuses, and methods for ophthalmic surgery are set forth in the appended claims. Illustrative embodiments are also provided to enable a person skilled in the art to make and use the claimed subject matter.

Some embodiments may comprise an apparatus that can mitigate the common issue of vitreous incarceration of cannulas. In more particular embodiments, a probe may be used during some surgical procedures to clear tissue from a cannula, such as an infusion cannula or protective cannula. For example, vitreous humor can block an infusion cannula during a vitrectomy, which can cause a softening or collapse of the eye as vacuum commences. One of the challenges surgeons may encounter is vitreous incarceration of an infusion cannula that can result in the blockage of infusion, which can cause a softening or collapse of the eye as vacuum commences. The probe can be inserted into the infusion cannula and positioned to cut the vitreous positioned just outside the cannula. The probe may be rotated as the vitreous is cut.

For example, the probe may comprise a cutting port and a sleeve that is sized so that it will only allow the probe to be inserted into the cannula as far as to allow the full cutting port to be exposed passed the distal end of the cannula. A surgeon can then operate the probe while rotating it to remove the immediate surrounding vitreous skirt so as to mitigate potential vitreous incarceration. Once cleared, the probe can be withdrawn from the cannula, the sleeve may be removed, and the vitrectomy continued.

In some embodiments, the cannula and probe may comprise an interference fit between a feature on the cannula and a feature on the probe to limit probe movement to a preferred position for cutting outside the cannula.

Additionally, or alternatively, a single slit valve may be disposed on the distal end of the cannula, which can stop vitreous being pulled through by a surgical tool (such as a trocar) as it is withdrawn from the eye.

More generally, a probe may comprise a tube, a cutting edge disposed within the tube, and a stop. The cannula may have a first end configured to receive the tube and a second end adapted for insertion into an eye. The stop can be configured to position the cutting edge adjacent to the second end of the cannula when the tube is inserted into the cannula.

In more particular embodiments, the stop may comprise a receptacle coupled to the tube, and the cannula may comprise a latch configured to engage the receptacle when the cutting edge is positioned adjacent to the second end of the cannula. In other embodiments, the stop may comprise a sleeve disposed around the tube, and the sleeve can be configured to contact the first end of the cannula when the cutting edge is positioned adjacent to the second end of the cannula.

In other aspects, some embodiments of an apparatus for clearing tissue from a cannula may comprise a housing, an actuator disposed within the housing, a first tube coupled to the housing and extending from the housing, and a second tube disposed within the first tube. The first tube may comprise a cutting port configured to be inserted into a first end of the cannula, and the second tube may comprise a cutting edge at least partially exposed through the cutting port. A stop can be configured to position the cutting edge adjacent to a second end of the cannula, and the actuator can be configured to oscillate the second tube within the first tube such that the cutting edge is operable to cut tissue entering the cutting port adjacent to the second end of the cannula.

In other example embodiments, a system for eye surgery may comprise a cannula having a first end and a second end, a power source, a controller, and a driver coupled to the power source and the controller. An actuator can be coupled to the driver, and a cutting edge can be coupled to the actuator. The second end of the cannula can be adapted for insertion into an eye, and the cutting edge can be configured to be inserted into the first end of the cannula. The system may further comprise a stop, wherein the stop is configured to position the cutting edge adjacent to the second end of the cannula when the cutting edge is inserted into the cannula. The controller can be configured to operate the driver to cause the actuator to oscillate the cutting edge to cut tissue adjacent to the second end of the cannula. In more particular embodiments, the system may comprise a tube enclosing the cutting edge, wherein the tube is configured to be inserted into the first end of the cannula.

Features, elements, and aspects described in the context of some embodiments may also be omitted, combined, or replaced by alternative features. Other features, objectives, advantages, and an example mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and an example mode of making and using some embodiments of the claimed subject matter. Like reference numbers represent like parts in the examples.

FIG. 1 is a functional block diagram of an example of a system for ophthalmic surgery.

FIG. 2 is a perspective view of an example of a probe that may be associated with some embodiments of the system of FIG. 1.

FIG. 3 is a perspective view of another example of the probe.

FIG. 4 is a detail view of a distal end of an example of a cutter that may be associated with some embodiments of the probe of FIG. 2 or FIG. 3.

FIG. 5 is a perspective view of an example of a cannula that may be associated with some embodiments of the system of FIG. 1.

FIG. 6 is another perspective view of the cannula of FIG. 5.

FIG. 7 is a schematic view of an example of the probe of FIG. 2 inserted into a first end of the cannula of FIG. 5.

FIG. 8 is a schematic view of an example of the probe of FIG. 3 inserted into the first end of the cannula of FIG. 5.

FIG. 9 is a schematic diagram of a method for using the system of FIG. 1.

FIG. 10 illustrates a perspective view of a vitrectomy probe with a dynamically adjustable stiffening sleeve and a control member, according to certain embodiments of the present disclosure.

FIGS. 11a-11b illustrate schematic cross-sectional side views of the vitrectomy probe of FIG. 1B, according to certain embodiments of the present disclosure.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position for ophthalmic surgery. However, as should be recognized by those skilled in the art, this frame of reference is merely a descriptive expedient rather than a strict requirement.

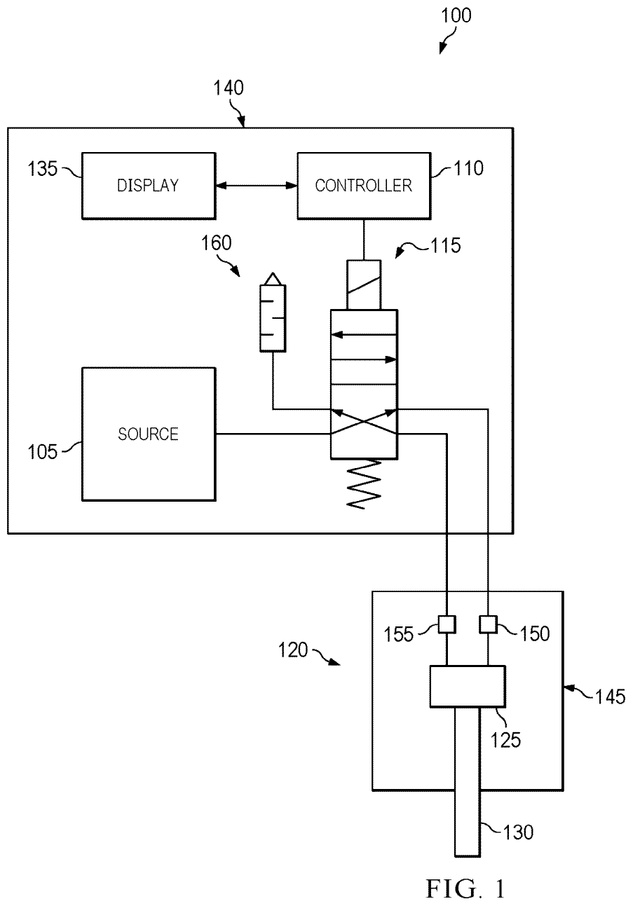

FIG. 1 is a functional block diagram of an example of a system 100 for ophthalmic surgery. As shown in FIG. 1, some examples of the system 100 may comprise a power source 105, a controller 110, a driver 115, and a probe 120. The probe 120 may comprise an actuator 125 and a cutter 130 coupled to the actuator 125. Some embodiments may also comprise an output device, such as a display 135. For example, the display 135 may be coupled to the controller 110, which can cause the display 135 to render information related to the operation and performance of the system 100 during a surgical procedure.

Some components of the system 100 may be housed within or used in conjunction with other components, such as sensors, processing units, alarm indicators, memory, databases, software, display devices, or user interfaces that further facilitate surgical procedures. For example, in some embodiments, the power source 105 may be combined with the controller 110, the driver 115, the display 135, and other components into a console housing 140. In some examples, the console housing 140 may be mobile, and may including wheels to facilitate movement. In some embodiments, the actuator 125 may be combined with other components into a probe housing 145. In the example of FIG. 1, the cutter 130 has a first end coupled to the actuator 125 within the probe housing 145 and a second end that extends out of the probe housing 145.

In general, components of the system 100 may be coupled directly or indirectly. For example, the power source 105 may be directly coupled to the driver 115 and may be indirectly coupled to the probe 120 through the driver 115. Components may also be coupled by virtue of physical proximity, being integral to a single structure, or being formed from the same piece of material. Components may also be coupled at varying levels of detail. For example, the driver 115 may be coupled to the actuator 125 and may also be coupled to the probe 120 by virtue of the actuator 125 being a component of the probe 120. Coupling may include fluid, mechanical, thermal, electrical, or chemical coupling (such as a chemical bond), or some combination of coupling in some contexts. For example, the driver 115 may be electrically coupled to the controller 110 and may be fluidly coupled to the actuator 125. Components may also include or comprise interfaces or ports to facilitate coupling and de-coupling other components.

An example of the power source 105 may be a pneumatic power source, which generally provides gas at a pressure greater than a local ambient pressure. In many cases, the local ambient pressure may also be the atmospheric pressure at which a surgical site is located. For example, a reservoir of compressed air or a compressor may be suitable as a source of pneumatic power. One or more fluid conductors may couple the power source 105 to other components. For example, the driver 115 may be an adjustable, directional on-off pneumatic driver, such as four-way on-off valve, which may be fluidly coupled to the power source 105. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components.

A controller, such as the controller 110, may be a microprocessor or computer programmed to operate one or more components of the system 100, such as the driver 115. In some embodiments, for example, the controller 110 may be a microcontroller, which generally comprises an integrated circuit containing a processor core and a memory programmed to control one or more operating parameters of the system 100, directly or indirectly. Operating parameters may include the pressure provided by the power source 105, or the pressure applied to the probe 120, for example. The controller 110 is also preferably configured to receive one or more input signals, such as a feedback signal, and programmed to modify one or more operating parameters based on the input signals. For example, the driver 115 may be configured to receive operating signals from the controller 110.

Additionally, the system 100 may include one or more sensors to measure operating parameters and provide feedback signals to the controller 110 indicative of the operating parameters. Sensors are generally known in the art as any apparatus operable to detect or measure a physical phenomenon or property, and generally provide a signal indicative of the phenomenon or property that is detected or measured. Preferably, signals from sensors are suitable as an input signal to the controller 110, but some signal conditioning may be appropriate in some embodiments. For example, the signal may need to be filtered or amplified before it can be processed by the controller 110. Typically, the signal is an electrical signal, but may be represented in other forms, such as an optical signal.

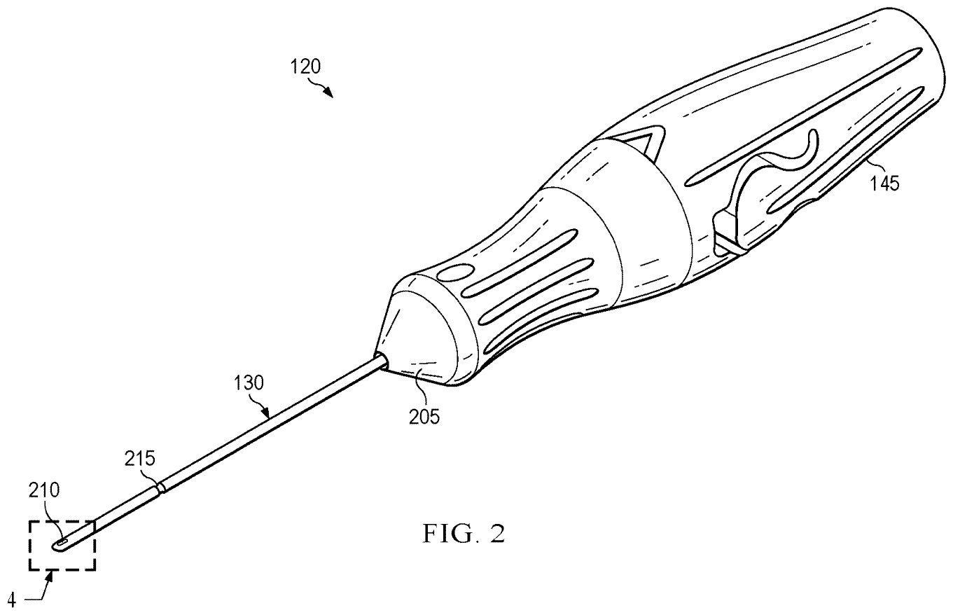

FIG. 2 is a perspective view of an example of the probe 120, illustrating additional details that may be associated with some embodiments. For example, the probe housing 145 of FIG. 2 includes a distal housing end 205, which can be tapered in some embodiments to facilitate positioning between a thumb and forefinger during use of the probe 120. Thus, the probe 120 may be supported at the purlicue between the thumb and forefinger. The probe 120 may comprise a variety of other supportive features and aspects. As shown in the example of FIG. 2, the cutter 130 can extend from the probe housing 145 and may have a cutting port 210 and a stop 215 disposed near a distal end. In this context, a stop refers to a mechanical or structural means to resist or arrest the movement of the cutter 130. For example, the stop 215 of FIG. 2 is a notch, groove, or other receptacle coupled to the cutter 130 between probe housing 145 and the cutting port 210.

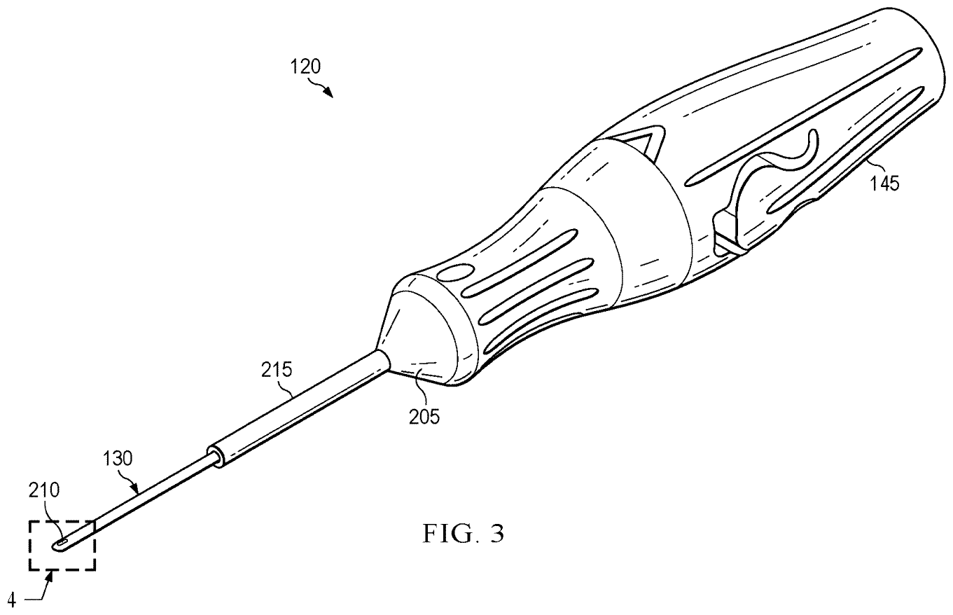

FIG. 3 is a perspective view of another example of the probe 120, illustrating additional details that may be associated with some embodiments. For example, the stop 215 of FIG. 3 is a sleeve disposed around the cutter 130. The sleeve may be positioned between the probe housing 145 and the cutting port 210. The sleeve may be constructed of a rigid material, or otherwise configured to provide rigidity. In some examples, the sleeve may be removable. Additionally, some embodiments of the sleeve may be disposable. For example, some embodiments of the sleeve may be comprised of a rigid plastic, which can be disposable or recyclable.

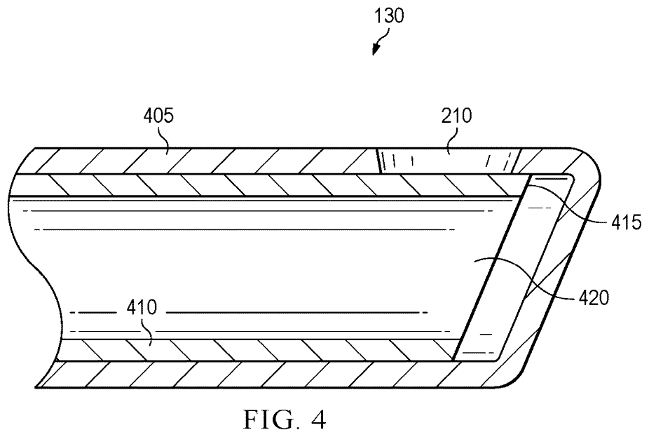

FIG. 4 is a detail view of a distal end of an example of the cutter 130, illustrating additional details that may be associated with some embodiments. For example, the cutter 130 of FIG. 4 comprises a first tube 405 and a second tube 410, which can extend from the probe housing 145 (FIG. 1). The first tube 405 and the second tube 410 may both be cylindrical tubes with a hollow bore. The first tube 405 may be coupled to the probe housing 145, and the second tube 410 may be coupled to the actuator 125 (see FIG. 1). The second tube 410 may comprise a cutting edge 415 on the distal end and may be disposed within the first tube 405. The second tube 410 and the cutting edge 415 may be at least partially exposed through the cutting port 210. In some embodiments, the second tube 410 may additionally have an aspiration port 420.

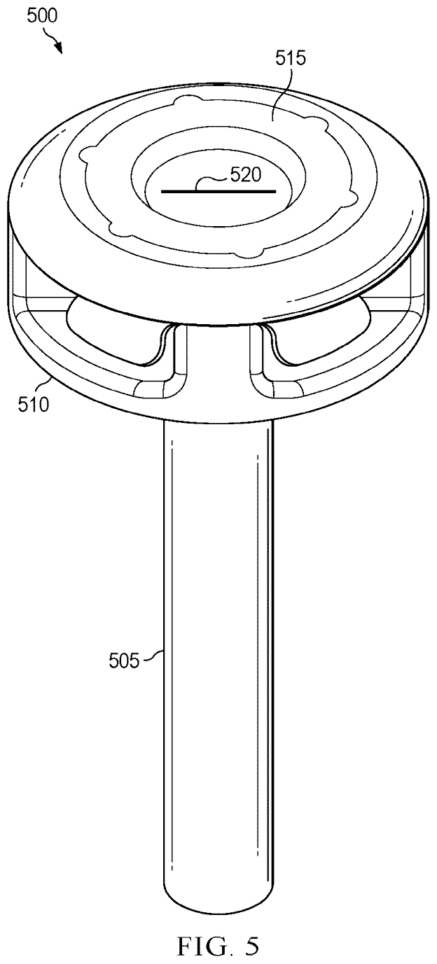

FIG. 5 is a perspective view of an example of a cannula 500 that may be associated with some embodiments of the system 100. For example, the cannula 500 may be configured for insertion into an eye to facilitate insertion and removal of instruments, such as the probe 120. Some examples of the cannula 500 may include a sleeve 505 and a hub 510. In some examples, a seal 515 may be coupled to the hub 510. For example, the seal 515 may be disposed at least partially over the hub 510 to form an over-molded seal. In some embodiments, the cannula 500 may comprise a valve 520. For example, the valve 520 may be a slit valve disposed at one end of the cannula 500, such as in an exposed surface of the seal 515, which may be normally closed and configured to be opened by surgical tools inserted into the cannula 500. In the absence of a surgical instrument, the seal 515 and the valve 520 may inhibit fluid flow through the seal 515.

The seal 515 may be made of an elastomer, such as silicone. In some embodiments, the seal 515 may be attached to the hub 510 to inhibit rotation of the seal 515 relative to the hub 510. In some embodiments, a friction fit may secure the seal 515 to the hub 510. Other attachments are also contemplated, such as an adhesive attachment.

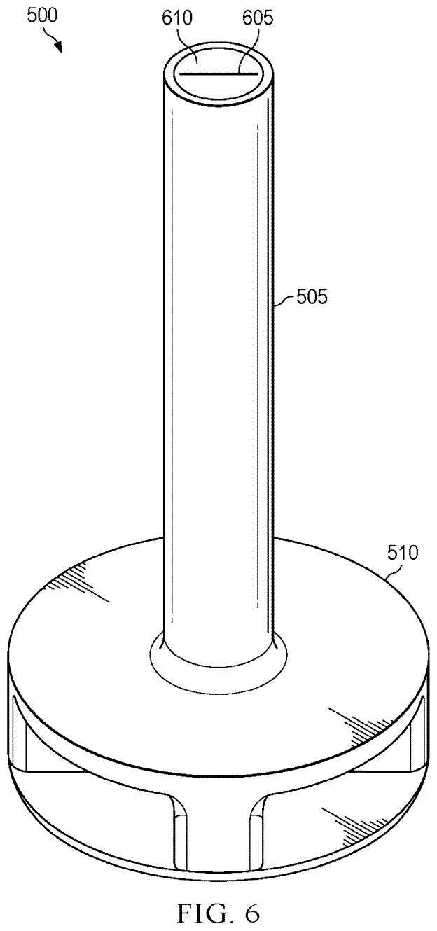

FIG. 6 is a perspective view of an example of a cannula 600, illustrating additional details that may be associated with some embodiments. As illustrated in the example of FIG. 6, a second end of the cannula 600 may comprise a valve 605. The cannula 600 may be similar to the cannula 500 in many respects, and the valve 605 may be similar to the valve 520. For example, the valve 605 may be a slit valve disposed in a seal 610, which may be coupled to the sleeve 505, near the end of the cannula 500 opposite the valve 520 (not shown in FIG. 6). In some embodiments, the valve 605 may be concave to facilitate the passage of soft tipped instruments. Additionally, or alternatively, the position of the valve 605 may be adjusted relative to the second end of the cannula 500.

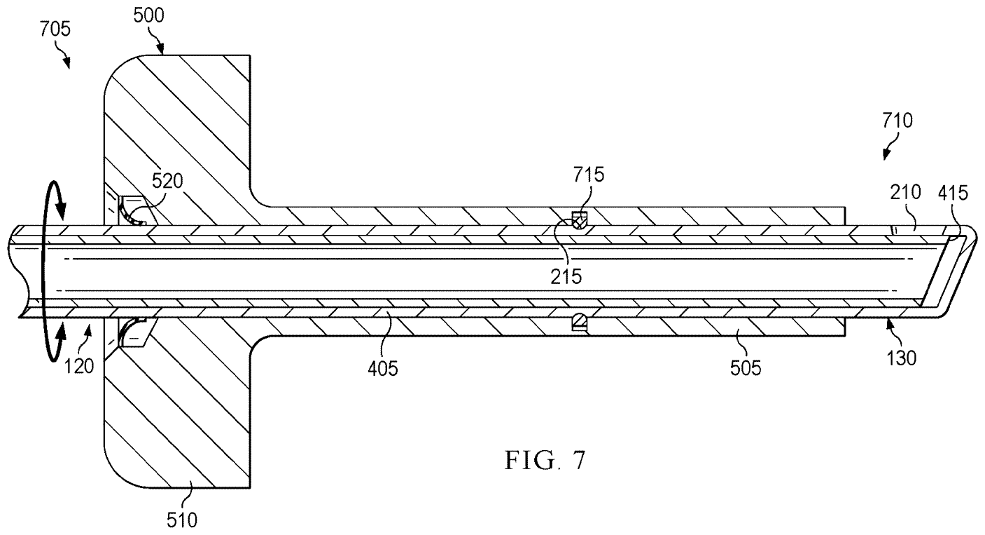

FIG. 7 is a schematic view of an example of the probe 120 of FIG. 2 inserted into a first end 705 of the cannula 500 of FIG. 5. As illustrated in the example of FIG. 7, the cutter 130 may be inserted through the first end 705, and the stop 215 may be configured to position the cutting edge 415 adjacent to the second end 710 of the cannula 500. For example, the cutting port 210 may be inserted through the valve 520 and an interference fit may facilitate positioning the cutter 130 in the sleeve 505 so that the cutting port 210 and the cutting edge 415 are disposed adjacent to the second end 710. In some embodiments, an interference fit may be facilitated by a tab, pawl, ball, dog, or other latch mechanism on the sleeve 505, which can be configured to engage the stop 215 on the cutter 130. As illustrated in the example of FIG. 7, the sleeve 505 comprises a latch 715 configured to engage the stop 215 of cutter 130 and provide resistance against further insertion of the cutter 130. The interference fit can allow the cutter 130 to be rotated within the sleeve 505 while maintaining the longitudinal position of the cutting edge 415 relative to the second end 710.

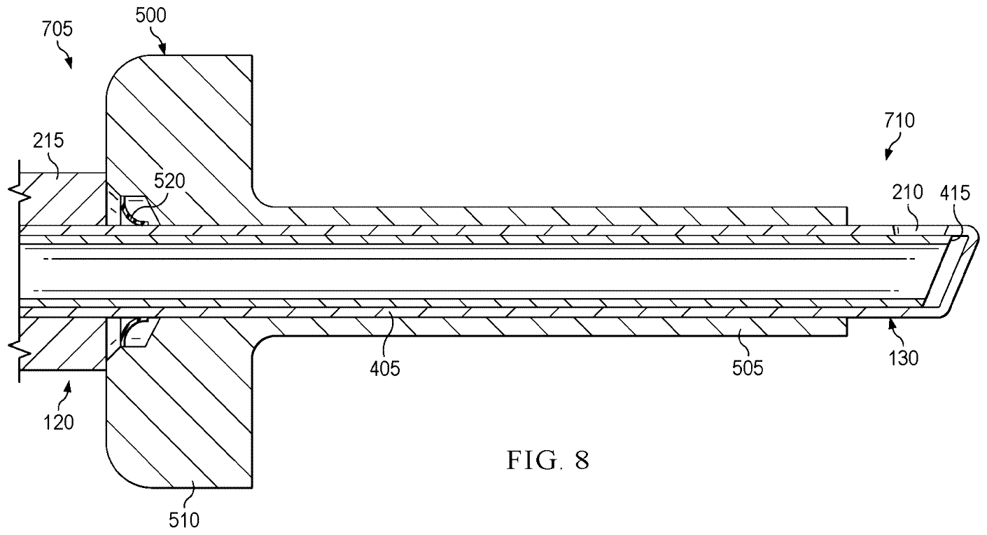

FIG. 8 is a schematic view of an example of the probe 120 of FIG. 3 inserted into the first end 705 of the cannula 500 of FIG. 5. As illustrated in the example of FIG. 8, the cutter 130 may be inserted through the first end 705, and the stop 215 may be configured to position the cutting port 210 adjacent to a second end 710 of the cannula. For example, the cutting port 210 may be inserted through the valve 520 into the sleeve 505 until the stop 215 contacts the hub 510 adjacent to the second end 710. The stop 215 can be sized so that the hub 510 allows the cutter 130 to be inserted only as far as to allow the cutting port 210 to be exposed past the second end 710 and prevents further insertion through the sleeve 505.

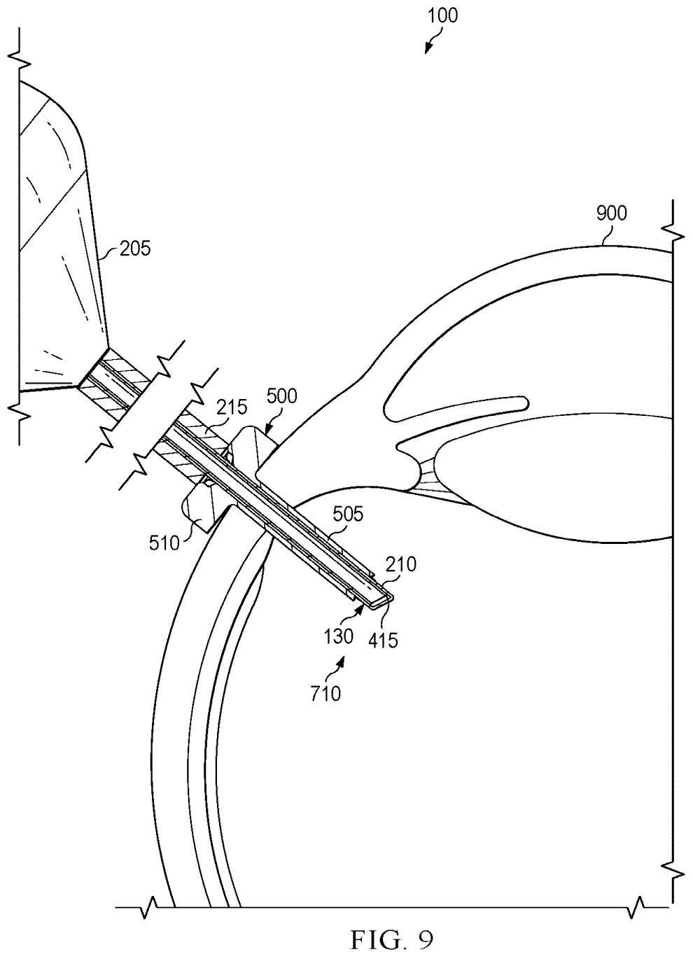

FIG. 9 is a schematic diagram of a method for using the system 100 with an eye 900. Generally, different types of cannulas may be used for different purposes. For example, in ophthalmic surgery, an infusion cannula may be used for administration of therapeutic fluids, gases or silicone oil to a patient's eye. To use an infusion cannula, a surgeon can make an incision in the eye and insert the cannula into the incision up to the hub, which acts as a stop, preventing the cannula from entering the eye completely. For example, a trocar can be used with the cannula 500 to make an incision into the eye 900, allowing the cannula 500 to be inserted into the eye 900 until the hub 510 contacts the eye 900. The trocar can be removed while leaving the cannula 500 in place. The hub 510 can be coupled to a tube, such as a plastic tube, through which fluids can be administered to the eye 900. An infusion cannula may be used during vitrectomy, which is a surgical procedure where the vitreous humor gel that fills the eye cavity is removed to provide better access to the retina. For example, fluid can be infused into an eye to maintain pressure in the eye as vitreous humor is removed. Other types of cannulas may also include cannulas that are inserted into an incision, made in the eye, to protect the incision's sidewalls from repeated contact by instruments that are inserted into and removed from the cannula. For example, protective cannulas can provide access to the eye to infuse or place other medications, devices, or illumination instruments within the eye.

During some surgical procedures, tissue from within the eye may block a cannula. For example, vitreous humor can block a cannula during a vitrectomy, which can cause a softening or collapse of the eye as vacuum commences. The system 100 can be used to mitigate this common issue of vitreous incarceration of cannulas. For example, to prevent or clear such a blockage, the cutter 130 may be inserted into the cannula 500, as illustrated in the example of FIG. 9. As described above, the stop 215 can cause the cutting port 210 and the cutting edge 415 to be positioned in the eye 900, just outside the sleeve 505 and adjacent to the second end 710. The phrase “just outside” is meant to cover the cutting port being positioned completely outside the sleeve 505 by a clearance distance between a proximal edge of the cutting port and a distal edge of the sleeve 505 approximately in a range between 0.5 mm to 5 mm, such as between 1 mm and 3 mm, such as 2 mm. Other clearance distances may also be used to be sufficient to clear the vitreous around distal end of the sleeve 505. The actuator 125 can be configured to oscillate the cutting edge 415 to cut tissue entering the cutting port 210.

For example, referring generally to FIG. 1, the controller 110 may operate the power source 105 and the driver 115 to alternately direct power to the first port 150 and the second port 155 to operate the actuator 125. In some embodiments, the power source 105 may provide pneumatic power to the driver 115, and the controller 110 may alternate the positions of the driver 115 very rapidly to provide pneumatic pressure to the first port 150 and the second port 155. Other embodiments of the system 100 may include more than one driver. For example, the system 100 may include two pneumatic drivers, wherein one may be coupled to the first port 150 and the other may be coupled to the second port 155. Such embodiments may operate similar to the manner described, with the drivers being configured to independently receive operating signals from the controller 110.

In some examples, the driver 115 may have a solenoid that operates to move the driver 115 between positions. For example, the power source 105 may be configured to deliver pneumatic power to the driver 115, and the driver 115 may be in a first position to provide pneumatic pressure to the first port 150 and to vent pneumatic pressure from the second port 155. In this position, pneumatic pressure may pass from the power source 105, through the driver 115, and to the first port 150, where the pneumatic pressure can operate the actuator 125. Pneumatic pressure at the second port 155 may pass through the driver 115 and then be exhausted. In some examples, the system 100 may include a muffler 160, and the pneumatic pressure may pass through the muffler 160 before being exhausted. In a second position, the driver 115 may allow pneumatic pressure to pass from the power source 105 to the second port 155, where the pneumatic pressure can operate the actuator 125. In the second position, pneumatic pressure at the first port 150 may vent through driver 115 and then be exhausted.

In some embodiments, the controller 110 may cause the second tube 410 to oscillate within the first tube 405. For example, the second tube 410 may be driven by air pressure directed to the actuator 125. If air pressure is increased at the first port 150, the actuator 125 may move the second tube 410 in a first direction relative to the first tube 405, thereby moving the cutting edge 415 in the first direction, which can cut any vitreous material or other tissue which may have been aspirated into the cutting port 210. The tissue may be aspirated through the aspiration port 420. Venting the pressure at the first port 150 and increasing the pressure at the second port 155 may move the second tube 410 in a second direction.

This process can cause the cutting edge 415 to cut tissue in the eye 900 adjacent to the second end 710 of the cannula 500 of FIG. 9, which can then be aspirated through the cutter 130. The cutter 130 may be rotated within the cannula 500 to remove tissue around the cannula 500. The cutter 130 may then be removed from the cannula 500, allowing other tools or an infusion line to be inserted into and/or coupled to the cannula 500 as appropriate. In some embodiments, the stop 215 may be removed from the probe 120. For example, if the stop 215 is a sleeve, such as in FIG. 3, the sleeve may be removed. In some examples, the valve 605 (FIG. 6) may also reduce or substantially prevent tissue being pulled through the sleeve 505 when the trocar, the cutter 130, or other instruments are removed from the cannula 500. Additionally, the probe 120 may then be used to perform other functions, such as a vitrectomy procedure.

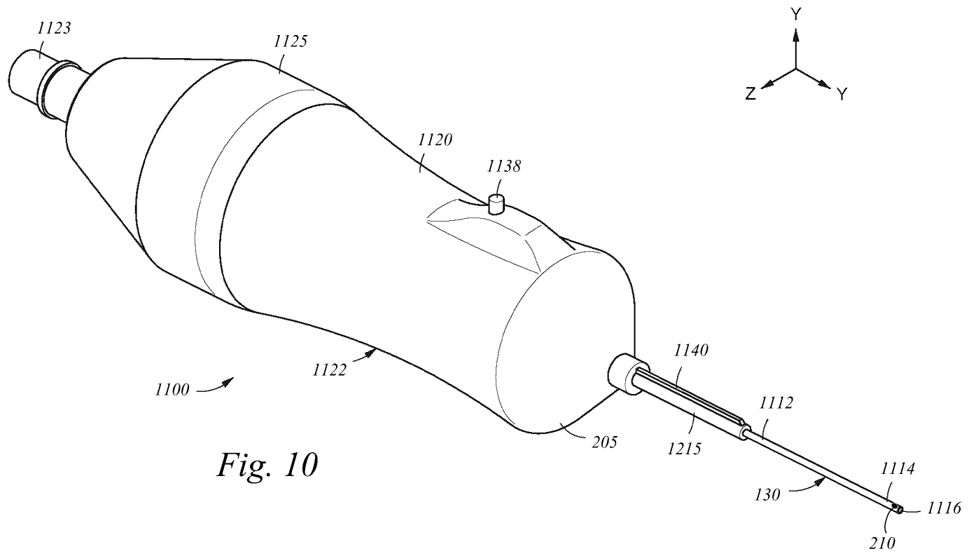

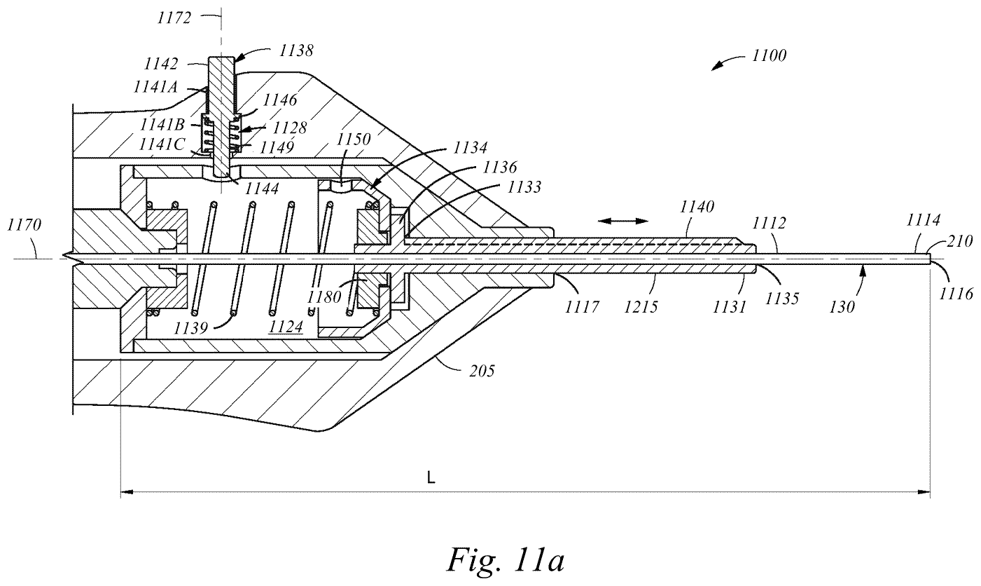

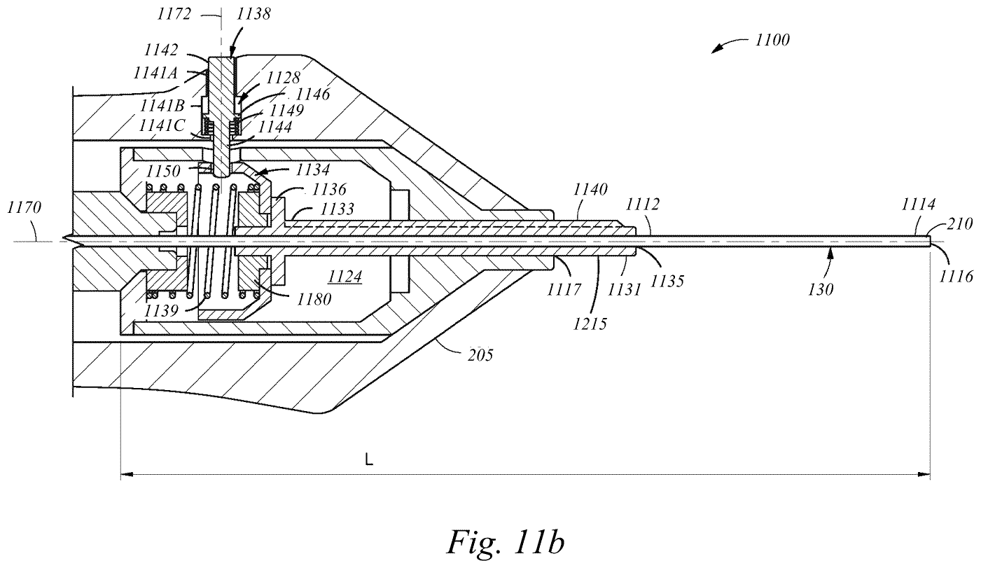

FIG. 10 illustrates a perspective view of a vitrectomy probe 1100 with a dynamically adjustable stop 1215, according to certain embodiments. The stop 215 shown in FIGS. 8-9 may be a dynamically adjustable stop 1215 that can cause the cutting port 210 and the cutting edge 415 (see FIG. 9) to be positioned in the eye 900, just outside the sleeve 505 and adjacent to the second end 710. The cutting edge 415 can be oscillated to cut tissue entering the cutting port 210. As depicted in FIG. 10, the instrument 1100 comprises a cutter 130 or needle (referred to hereinafter as a “cutter”) and a base unit 1120. The cutter 130 includes a proximal portion 1112 and a distal portion 1114 which terminates distally at a distal end 1116. In some embodiments, the proximal portion 1112 extends through a substantial portion of an interior chamber (e.g., an interior chamber 1124 FIGS. 11A and 11B) of the base unit 1120.

In one example, the cutter 130 is an elongated cutting member of a vitrectomy probe. For example, the cutter 130, which may be aspirating or non-aspirating, may be inserted into a cannula for performance of vitreous surgery. The cutter 130 may comprise a hollow tube having, e.g., a diameter less than about 20 gauge. For example, the cutter 130 has a diameter less than about 23 gauge, such as a diameter less than about 25 gauge. In certain embodiments, the cutter 130 has a diameter of approximately 27 gauge. In further examples, the cutter 130 may include an illumination device, a laser guide, a suction device, forceps, scissors, retractors, or other suitable devices disposed therein or coupled thereto.

Generally, the cutter 130 is formed of a material suitable for minimally invasive surgical procedures, such as vitreoretinal surgeries that involve removal of the vitreous in the eye, or other surgical procedures. For example, the cutter 130 is formed of surgical grade stainless steel, aluminum, or titanium.

The cutter 130 is partially and longitudinally disposed through a distal housing end 205 of the base unit 1120 adjacent the proximal portion 1112 of the cutter 130 and may be directly or indirectly attached thereto within the interior chamber 1124 of the base unit 1120. In certain embodiments, the base unit 1120 is a handpiece having an outer surface 1122 configured to be held by a user, such as a surgeon. For example, the base unit 1120 may be contoured to substantially fit the hand of the user. In some embodiments, the outer surface 1122 may be textured or have one or more gripping features formed thereon, such as one or more grooves and/or ridges.

In certain embodiments, the base unit 1120 may house at least a portion of a drive mechanism operable to reciprocate the cutter 130 within and relative to the base unit 1120. In one example, the drive mechanism may be a pneumatic drive mechanism including a diaphragm. The base unit 1120 may further provide one or more ports 1123 at a proximal end 1125 thereof for one or more supply lines to be routed into the interior chamber 1124. For example, the one or more ports 1123 may provide a connection between the base unit 1120 and a vacuum source for aspiration. In another example, the one or more ports 1123 provide a connection to a pneumatic, hydraulic, or electrical power source to operate the drive mechanism, an illumination device, a laser, or other suitable device within or coupled to the base unit 1120.

The instrument 1100 further includes a stop 1215 slidably coupled to and substantially surrounding at least a portion of the cutter 130. The stop 1215 is adjustable relative to the cutter 130, enabling a user to position the stop 1215 (e.g., a distal end 1131 of the stop 1215) at different points along a length L of the cutter 130 exterior to the base unit 1120.

In some embodiments the stop 1215 is generally a cylindrical and hollow tube substantially surrounding the cutter 130 at or near the proximal portion 1112. Similar to the cutter 130, the stop 1215 is formed of a material suitable for minimally invasive surgical procedures, such as vitreoretinal surgeries and other surgical procedures. In some embodiments, the stop 1215 is formed of a metallic material, such as surgical grade stainless steel, aluminum, or titanium. In other embodiments, the stop 1215 is formed of a composite material, such as a polymer composite material or a ceramic composite material.

As seen in FIGS. 11A-11B, an inner cavity 1135 of the stop 1215 is sized to accommodate an outer diameter of the cutter 130 while also permitting the stop 1215 to be readily moved along the cutter 130. Thus, an inner diameter or width of the stop 1215 is greater than the outer diameter of the cutter 130 and enables a sliding fit. In one embodiment, a radial clearance between the stop 1215 and the cutter 130 is between about 0.00020 inches and about 0.00060 inches, such as between about 0.00025 inches and about 0.00050 inches. For example, the radial clearance between the stop 1215 and the cutter 130 is between about 0.00030 inches and about 0.00040 inches, such as about 0.00035 inches. Further, the inner dimensions of the stop 1215 may be uniform from the distal end 1131 to the proximal end 1133 to enable uniform stabilization of the cutter 130 throughout the inner cavity of the stop 1215.

Along with the cutter 130, the stop 1215 is disposed through the base unit opening 1117 of the distal housing end 205 of the base unit 1120 and has a proximal end 1133 disposed in the interior chamber 1124 of the base unit 1120. As shown, the stop 1215 includes an annular flange (e.g., flange 1136) disposed at its proximal end 1133 within the interior chamber 1124. In other embodiments, the flange 1136 is disposed more axially along a length of the stop 1215. The flange 1136 is configured to prevent the stop 1215 from completely sliding through the base unit opening 1117 and out of the base unit 1120. Thus, the flange 1136 acts as an anchor in one capacity. The flange 1136 provides a coupling surface between the stop 1215 and a de-coupler 1134, which is further coupled to a stiffener biasing device 1139 (e.g., a spring such as a compression spring). In some embodiments, the stop 1215 may include a reduced diameter nose 1143. The reduced diameter nose 1143 may be able to extend further into a cannula in an eye of a patient.

The stiffener biasing device 1139 applies a biasing force against the de-coupler 1134 and thus the stop 1215 in a distal direction (e.g., towards the distal housing end 205) to bias the stop 1215 towards a protracted position. Thus, without an application of a force in an opposite, proximal direction (e.g., towards the proximal end 1125 in FIG. 10), the stop 1215 is constantly disposed in the protracted position. During use, the cutter 130 may be inserted into an insertion cannula with a hub (e.g., including a valve). Upon a distal end 1131 of the stop 1215 reaching the hub of the insertion cannula, the cutter 130 may stop and be correctly positioned to cause the cutting port 210 and the cutting edge 415 (see FIG. 9) to be positioned in the eye 900, just outside the sleeve 505 and adjacent to the second end 710. In some embodiments, the cutting port may be completely outside the sleeve 505 by a clearance distance between a proximal edge of the cutting port and a distal edge of the sleeve 505 approximately in a range between 0.5 mm to 5 mm, such as between 1 mm and 3 mm, such as 2 mm. Other clearance distances may also be used to be sufficient to clear the vitreous around distal end of the sleeve 505. The cutting edge 415 can be oscillated to cut tissue entering the cutting port 210.

In certain embodiments, the stop 1215 is sized to possess an axial length sufficient to cause the cutting port 210 and the cutting edge 415 (see FIG. 9) to be positioned in the eye 900, just outside the sleeve 505 and adjacent to the second end 710. For example, the stop 1215 may have an axial length between about 0.25 inches and about 1.75 inches, such as between about 0.30 inches and about 1.50 inches. For example, the stop 1215 may have an axial length between about 0.50 inches and about 1.25 inches.

In certain embodiments, the stop 1215 has a uniform outer diameter from the distal end 1131 to the proximal end 1133. Having a uniform outer diameter enables a substantial length of the stop 1215 to be reciprocated through the base unit opening 1117 without forming an air gap therebetween. However, other shapes and morphologies of the stop 1215 are also contemplated. For example, in some embodiments, the stop 1215 comprises a square, rectangular, or polygonal tube. In further embodiments, the stop 1215 may have a non-uniform outer diameter. For example, the stop 1215 may have an outer diameter having one or more dimensions following a step-wise or gradual delta.

In some embodiments, the actuation mechanism may include a biasing device 1139, a de-coupler 1134, and an annular flange 1136 integral with or affixed to the stop 1215 such that the biasing device is configured to apply a biasing force, through the de-coupler 1134, against the annular flange 1136 of the stop 1215 in the distal direction. In some embodiments, the de-coupler 1134 and the stop 1215 are separate components that are biased toward each other by, for example, biasing device 1139 (such as a spring). The de-coupler 1134 may contact annular flange 1136 due to the biasing device 1139 biasing the de-coupler 1134 toward the annular flange 1136 and/or due to external forces on the stop 1215 pushing the annular flange 1136 (which may be integral with or attached to the stop 1215) toward the de-coupler 1134. In some embodiments, the de-coupler 1134 and annular flange 1136 may be otherwise not attached to each other to allow relative movement between the de-coupler 1134 and annular flange 1136.

FIG. 11B illustrates a perspective view of a vitrectomy probe with a dynamically adjustable stiffening sleeve and a control member 1138. In some embodiments, the vitrectomy probe further has a de-coupler 1134 and a control member 1138 configured to selectively allow the stop 1215 to be locked in a retracted position. In certain embodiments, the position of the stop 1215 is locked in place using the control member 1138 as described below in relation to FIGS. 11A-B. For example, after extended stop 1215 abuts the cannula to cause the cutting port 210 and the cutting edge 415 (see FIG. 9) to be positioned in the eye 900, just outside the sleeve 505 and adjacent to the second end 710, the user may clear the vitreous at the entrance of the second end 710. The stop may then be compressed or pushed into the vitrectomy probe until the control member 1138 engages the hole 1150 of the decoupler to lock the sleeve in the retracted position for the rest of the procedure. In some embodiments, the surgeon may leave the stop 1215 in an extended position during the surgery. Accordingly, a user may selectively adjust the level of stiffness of the cutter 130 by re-positioning the stop 1215 relative to the distal end 1116, thereby manipulating the amount of support provided to the cutter 130 and stabilizing the instrument 1100 during use thereof.

In some embodiments, the stop 1215 includes a keying feature 1140 configured to operatively engage a base unit opening (e.g., a base unit opening 1117 in FIG. 11A) in the distal housing end 205 of the base unit 1120 to prevent rotation of the stop 1215 as further described in FIG. 11A. As shown, the keying feature 1140 is a protrusion of the stop 1215 with a rectangular-shaped cross-section but may be other shapes in other embodiments, such as a semi-circle or triangle. Note that although FIG. 10 shows a keying feature 1140, in certain embodiments (for example, as seen in FIG. 10), a keying feature 1140 is not used.

FIGS. 11A-B illustrate schematic cross-sectional views of the instrument 1100 with the stop 1215 positioned at different points along a length L of the cutter 130. Therefore, FIGS. 11A-B are herein described together with FIG. 10 for clarity. When the stop 1215 is positioned at different points along the length L, the keying feature 1140 operatively engages the base unit opening 1117 and prevents the stop 1215 from rotating. This beneficially ensures the opening of the de-coupler 1134 (referred to as de-coupler opening) does not rotate. A dashed line is shown between a cylindrical body of the stiffener and the keying feature 1140 in FIGS. 11A-B, and later figures including the stop 1215, to emphasize that the keying feature 1140 protrudes from the rest of the stop 1215.

In some embodiments, the stiffener biasing device 1139 applies a biasing force against the de-coupler 1134 and thus the stop 1215 in a distal direction (e.g., towards the distal housing end 205) to bias the stop 1215 towards a protracted position along the length L of the cutter 130, as shown in FIG. 11A. During use, the cutter 130 may be inserted into an insertion cannula with a hub (e.g., including a valve), at a desired depth along the length L selected by the user. Upon a distal end 1131 of the stop 1215 reaching the hub of the insertion cannula, the user may further press the instrument 1100 towards the hub to drive the cutter 130 deeper therein. Application of a force against the hub greater than that of the force provided by the stiffener biasing device 1139 will cause the stop 1215 to retract into the base unit 1120 (shown in FIG. 11B), allowing a greater portion of the cutter 130 to enter the eye. Once retracted, the stop 1215 can be locked in the retracted position by the control member 1138.

As shown in FIG. 11B, the position of the stop 1215 can be locked or maintained through the interaction of the control member 1138 and the de-coupler 1134. For example, a surgeon may press the control member 1138 radially-inward towards the de-coupler 1134, thereby causing the control member 1138 and the de-coupler 1134 to engage for locking the stop 1215 in position. More specifically, the control member 1138 operationally engages the de-coupler 1134 through an opening 1150 in the de-coupler 1134. The control member 1138 may be a button, knob, switch, toggle, or any other suitable device capable of being actuated by a user. As shown, the de-coupler opening 1150 is a through hole.

As depicted in FIGS. 11A-B, the control member 1138 includes a head 1142, a protrusion (e.g., a shaft 1144), and a flange 1146, wherein the head 1142 and the shaft 1144 are disposed at opposite ends of the control member 1138 and the flange 1146 is in between. The control member 1138 is partially disposed within a cutout 1128 (e.g., a channel or an opening) formed in the base unit 1120. The cutout 1128 includes multiple-sized passageways 1141 configured to accommodate the features of the control member 1138. For example, the head 1142 is disposed in a first passageway 1141A, the flange 1146 is disposed in a second passageway 1141B, and the shaft 1144 is at least partially disposed in a third passageway 1141C. The flange 1146 operatively engages the second passageway 1141B to guide the control member 1138 through the cutout 1128 and ensure the control member 1138 remains coupled to the base unit 1120. The cutout 1128 runs substantially perpendicular to a longitudinal axis 1170 of the cutter 130 (referred to as a probe longitudinal axis) and enables bidirectional pushing of the control member 1138 along a perpendicular axis 1172 thereof. The perpendicular axis 1172 may be referred to as a longitudinal axis of and with respect to the control member (e.g., a control member longitudinal axis) that is different from the probe longitudinal axis of the cutter 130.

As shown, a control member biasing device 1149 (e.g., a spring) is disposed in the second passageway 1141B to bias the control member 1138 in a radially outward direction along the perpendicular axis 1172. The control member biasing device 1149 applies a control member biasing force against the control member 1138 in a direction substantially parallel to the perpendicular axis 1172 and radially-outward from the de-coupler 1134 to bias the control member 1138 towards a protracted position as shown in FIG. 11A. Thus, without an application of a force in an opposite direction to retract the control member 1138, as shown in FIG. 11B, the control member 1138 is constantly disposed in the protracted position. Further, the control member biasing device 1149, the passageways 1141, and the head 1142 of the control member 1138 are sized and configured to ensure the shaft 1144 never touches the stiffener biasing device 1139 when the control member 1138 is retracted.

During use, the stop 1215 and the de-coupler 1134 are positioned at a retracted point along the length L of the cutter 130 as shown in FIG. 11B. The head 1142 of the control member 1138 is depressed by, e.g., a surgeon, and the shaft 1144 operatively engages the de-coupler opening 1150 in the de-coupler 1134, and thus, the stop 1215. Accordingly, depressing the control member 1138 into the de-coupler opening 1150 holds the stop 1215 in a retracted position, beneficially withholding the force from the stiffener biasing device 1139 while the control member 1138 is depressed. Releasing the control member 1138 pushes the control member 1138 towards the protracted position and thus operatively disengages the de-coupler 1134. The force from the stiffener biasing device 1139 returns the stop 1215 to the protracted position as shown in FIG. 11A.

Generally, the control member 1138 may be formed of a metallic or composite material. In some embodiments, the control member 1138 is formed of stainless steel, aluminum, or titanium. In other embodiments, the control member 1138 is formed of a polymer composite material or ceramic composite material.

The configurations of stop 1215, the de-coupler 1134, the control member 1138, and the biasing devices 1139 and 1149 are only exemplary and thus should not be considered limiting. Additional embodiments and configurations for different actuation mechanisms are further described below.

As shown in FIG. 11A, a nut 1180 couples the stop 1215 to the de-coupler 1134. In other embodiments, the de-coupler 1134 is a direct extension of the stop 1215. That is, the de-coupler 1134 and the stop 1215 are a single, integral component. In other embodiments, the de-coupler 1134 and the stop 1215 are separate components that are biased toward each other by, for example, biasing device 1139. In some embodiments, the de-coupler 1134 and the stop 1215 are coupled to one another by one or more coupling mechanisms and/or adhesives. In other embodiments, the de-coupler 1134 and the stop 1215 may be snap-fit together.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A system for eye surgery, the system comprising:
a probe comprising:
a tube,
a cutting edge disposed within the tube, and
a stop; and
a cannula having a first end configured to receive the tube and a second end adapted for insertion into an eye;
wherein the stop is configured to position the cutting edge adjacent to the second end of the cannula when the tube is inserted into the cannula.

2. The system of claim 1, wherein:
the stop comprises a receptacle coupled to the tube; and
the cannula comprises a latch configured to engage the receptacle when the cutting edge is positioned adjacent to the second end of the cannula.

3. The system of claim 1, wherein:
the stop comprises a sleeve disposed around the tube; and
the sleeve is configured to contact the first end of the cannula when the cutting edge is positioned adjacent to the second end of the cannula.

4. The system of claim 1, wherein:
the second end of the cannula comprises a valve; and
the valve is normally closed and configured to be opened when the tube is inserted through the second end.

5. The system of claim 1, wherein:
the stop comprises a sleeve disposed around the tube;
the sleeve is configured to contact the first end of the cannula when the cutting edge is positioned adjacent to the second end of the cannula;
the second end of the cannula comprises a slit valve; and
the slit valve is normally closed and configured to be opened when the tube is inserted through the second end.

6. The system of claim 1, wherein the stop is spring biased and the probe further comprises a de-coupler and a control member configured to selectively allow the stop to be locked in a retracted position.

7. An apparatus for clearing tissue from a cannula, the apparatus comprising:
a housing;
an actuator disposed within the housing;
a first tube coupled to the housing and extending from the housing, the first tube comprising a cutting port configured to be inserted into a first end of the cannula;
a second tube disposed within the first tube, the second tube comprising a cutting edge at least partially exposed through the cutting port; and
a stop configured to position the cutting edge adjacent to a second end of the cannula;
wherein the actuator is configured to oscillate the second tube within the first tube such that the cutting edge is operable to cut tissue entering the cutting port adjacent to the second end of the cannula.

8. The apparatus of claim 7, wherein:
the stop comprises a sleeve disposed around the first tube and positioned between the housing and the cutting port; and
the sleeve is configured to contact the first end of the cannula when the cutting edge is positioned adjacent to the second end of the cannula.

9. The apparatus of claim 7, wherein:
the stop comprises a receptacle coupled to the first tube; and
the receptacle is configured to engage the cannula when the cutting edge is positioned adjacent to the second end of the cannula.

10. A system for eye surgery, the system comprising:
a cannula having a first end and a second end;
a power source;
a controller;
a driver coupled to the power source and the controller;
an actuator coupled to the driver;
a cutting edge coupled to the actuator; and
a stop;
wherein:
the second end of the cannula is adapted for insertion into an eye,
the cutting edge is configured to be inserted into the first end of the cannula,
the stop is configured to position the cutting edge adjacent to the second end of the cannula when the cutting edge is inserted into the cannula, and
the controller is configured to operate the driver to cause the actuator to oscillate the cutting edge to cut tissue adjacent to the second end of the cannula.

11. The system of claim 10, wherein:
the second end of the cannula comprises a valve; and
the valve is normally closed and configured to be opened when the cutting edge is inserted through the second end.

12. The system of claim 10, further comprising a tube enclosing the cutting edge, the tube configured to be inserted into the first end of the cannula.

13. The system of claim 12, wherein:
the stop comprises a receptacle coupled to the tube; and
the cannula comprises a latch configured to engage the receptacle when the cutting edge is positioned adjacent to the second end of the cannula.

14. The system of claim 12, wherein:
the stop comprises a sleeve disposed around the tube; and
the sleeve is configured to contact the first end of the cannula when the cutting edge is positioned adjacent to the second end of the cannula.

15. The system of claim 12, wherein:
the stop comprises a sleeve disposed around the tube;
the sleeve is configured to contact the first end of the cannula when the cutting edge is positioned adjacent to the second end of the cannula;
the second end of the cannula comprises a slit valve; and
the slit valve is normally closed and configured to be opened when the tube is inserted through the second end.
